(19) United States
(12) Patent Application Publication
(10) Pub. No.: US 2014/0294994 A1
(43) Pub. Date: Oct. 2, 2014

(54) PHARMACEUTICAL COMPOSITION FOR ELIMINATION OF CANCER STEM CELLS

(71) Applicant: Ch-Ying Huang, Taipei City (TW)

(72) Inventor: Ch-Ying Huang, Taipei City (TW)

(21) Appl. No.: 14/354,873

(22) PCT Filed: Oct. 29, 2012
(86) PCT No.: PCT/CN2012/083698

§ 371 Date: Apr. 28, 2014

Related U.S. Application Data
(60) Provisional application No. 61/553,146, filed on Oct. 28, 2011.

(51) Int. Cl. A61K 31/5415; A61N 5/10; A61K 45/06

(52) U.S. Cl. CPC A61K 31/5415; A61K 45/06; A61N 5/10
USPC 424/649; 544/44; 514/225.8; 544/41; 514/226.2; 544/42; 514/225.5; 600/1

(57) ABSTRACT

The preset invention relates to use of antipsychotic phenothiazine derivative for eliminating cancer stem cells (CSCs) and/or preventing a cancer. The invention also provides a pharmaceutical composition for treating a cancer, and/or preventing or delaying cancer recurrence comprising trifluoperazine and an anti-cancer drug, such as gefitinib or cisplatin.

### FIELD OF THE INVENTION

The present invention relates to a pharmaceutical composition for elimination of cancer stem cells.

### BACKGROUND OF THE INVENTION

Recently, the cancer stem-like cell (CSC) hypothesis has drawn much attention. CSCs possess stem cell characteristics, including self-renewal, stress/drug resistance and enhanced migration, which may contribute to tumor recurrence, metastasis, and chemoresistance. Putative lung CSCs were first identified as CD133⁺/Oct4⁺/Nanog⁺ cells (Eramo A et al. Cell Death Differ 2008; 15:504-514) and isolated in established NSCLC cell lines (Pirozzi G et al. PloS one 2011; 6:e21548; and Leung E L et al. PloS one 2010; 5:e14062). Lung CSCs may share functional features with lung progenitor cells, including the ability to actively exclude the dye Hoechst 33342, which defines them as side population cells in flow cytometric assays (Storms R W et al. Blood 2000; 96:2125-2133), and displays high aldehyde dehydrogenase (ALDH) activity (Ginestier C et al. Cell Stem Cell 2007; 1:555-567). CSCs express high levels of ABCG2, a multidrug transporter, and demonstrate resistance to TKI treatment by modulating intracellular TKI concentrations (Ozvegy-Laczka C et al. Mol Pharmacol 2004; 65:1485-1495). Thus, targeting CSCs among cancer cell may be critical in overcoming drug resistance. For example, most advanced stage lung cancer patients receiving front-line chemotherapy experience disease progression (Pfister D G et al. J Clin Oncol 2004; 22:330-353). The poor outcome implies that current treatment modalities, such as surgery and chemo- or radiotherapy alone or in combination, are ineffective for the treatment or even the control of this disease. In advanced non-small cell lung cancer (NSCLC) patients with specific EGFR mutations, the outcome of treatment with EGFR-tyrosine kinase inhibitors (TKIs), such as gefitinib, is significantly better than with traditional chemotherapy drugs (Maemondo et al. N Engl J Med 362:2380-2388, 2010; and Mok et al. N Engl J Med 361:947-957, 2009). However, after EGFR-TKI treatment, almost all patients will eventually develop drug resistance after a period of few months and present with tumor recurrence at the primary site or metastases to distant organs. Among these patients, approximately 25% of patients will eventually develop brain metastasis (Ceresoli et al. Ann Oncol 15:1042-1047, 2004; Kim et al. Lung cancer 65:351-354, 2009; Wu et al. Lung cancer 57:359-364, 2007; and Heon et al. Clin Cancer Res 16:5873-5882, 2010). To date, there is no effective intervention once EGFR-TKI resistance and metastases occur. It is imperative to search for new drugs for this unmet clinical need.

### SUMMARY OF THE INVENTION

In the present invention, it is unexpectedly found that several antipsychotic phenothiazine derivatives possess anti-CSC effects, as evidenced by their ability to suppress tumor spheroid formation and down-regulate Wnt/β-catenin signaling. More importantly, when combined with genifinib or ciplatin, the antipsychotic as used in the instant invention was able to enhance treatment response and overcome drug resistance, which implies a great potential for the treatment of various cancers. Since an antipsychotic phenothiazine is accessible to brain via blood-brain barrier, the invention should be particularly beneficial for the treatment of cancers that metastasize to the brain, such as lung cancer where 25% of patients with EGFR mutation will eventually develop brain metastasis after EGFR-TKI treatment.

Therefore, in one aspect, the invention provides use of a compound having the structure of formula I in the manufacture of a medicament for eliminating cancer stem cells (CSCs):

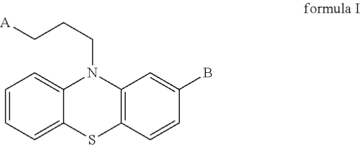

wherein the 10H-phenothiazine derivatives bearing an alkyl substituent, in which A is N(CH₃)₂, a N-methyl or N-ethyl piperazinyl group, a N-(hydroxyethyl)piperazinyl group, or a N-methyl piperidinyl group, and B is SCH₃, Cl, CF₃, or H. The aforementioned method also prevents a cancer in a subject in need thereof.

In another aspect, the present invention also provides a pharmaceutical composition for treating a cancer comprising a therapeutically effective amount of trifluoperazine and an anti-cancer drug.

In further aspect, the present invention also provides a pharmaceutical composition for preventing or delaying cancer recurrence comprising a therapeutically effective amount of trifluoperazine and an anti-cancer drug.

In yet aspect, also provided is use of a compound having the structure of formula I as aforementioned in the manufacture of a medicament for preventing a cancer.

### BRIEF DESCRIPTION OF THE DRAWINGS

The foregoing summary, as well as the following detailed description of the invention, will be better understood when read in conjunction with the appended drawing. In the drawings:

FIG. 1 provides the effects of trifluoperazine in inhibiting proliferation and inducing apoptosis of gefitinib-resistant NSCLC cells, wherein FIG. 1A provides the results of various NSCLC cells in 96-well plates that were treated with trifluoperazine for 48 hr, in which cell viability was measured by the MTT assay; FIG. 1B provides the results of CL141 cell line that was incubated with DMSO or the indicated concentrations of trifluoperazine for 48 hours, in which the numbers indicate the percentages of total cells in the corresponding quadrant; the bottom right quadrant is the early apoptotic cells, and the top right quadrant is late apoptotic cells; FIG. 1C shows the results of side population assay, in which the cancer stem-like side population was significantly decreased by trifluoperazine (5 μM), from 2.13% to 0.11% in CL141 cells, and from 1.95% to 0.06% in CL152 cells; FIG. 1D shows that the aldehyde dehydrogenase (ALDH)-positive subpopulation of cancer stem-like cells was reduced by trifluoperazine (5 μM), from 4.31% to 0.84% in CL141 cells, and from 3.73% to 1.08% in CL152 cells; and FIG. 1E shows that trifluoperazine dose-dependently activated apoptotic signaling in CL97 spheroids, including Bax, Bak, and cleaved PARP, caspase 3, and caspase 9, whereas the anti-apoptotic proteins Bcl-2, XIAP, and Mcl-1 were down-regulated. All values are the average of triplicate experiments with the S.D. indicated by the error bars, and there are statistically significant differences, for example, between treatment with and without trifluoperazine (*, P<0.05; **, P<0.01).

FIG. 2 provides the effects of trifluoperazine in inhibiting the capacity of lung cancer spheroid self-renewal, wherein FIGS. 2A and 2B respectively show the size and the number of CL83, CL141 and CL97 spheroids after treatment with trifluoperazine for 48 hr (n=3; **, P<0.01); FIG. 2C provides the images of CL141 colonies taken under phase microscopy (top panel) and the number of the colonies (bottom panel) calculated after two weeks of treatment with trifluoperazine, in which colonies containing >50 cells were counted and the number of colonies in the control group was set at 100% (n=3; **, P<0.01); FIG. 2D provides the expression of CD44 and CD133 in CL141 and CL97 cancer spheroids after being treated with different doses of trifluoperazine for 48 hr, in which the expression was evaluated by Western blot analysis, and β-actin served as an internal control; FIG. 2E provides immunostained images for CD133 and nuclei counterstaining (DAPI) of various spheroids at 48 hours after trifluoperazine (TFP) treatment, in which photomicrographs were taken at 40× magnification; FIG. 2F provides the expression of c-Myc, cyclin D1 and c-Met in CL97 cancer spheroids after being treated with different doses of trifluoperazine for 48 hr, in which the expression was evaluated by Western blot analysis, and β-actin served as an internal control; and FIG. 2G provides TCF/LEF transcription following treatment of CL141 cancer spheroids with different concentrations of trifluoperazine for 24 hours, in which cells were lysed before the TOPflash and FOPflash activities were recorded in a luminometer (n=3; *, P<0.05; **, P<0.01).

FIG. 3 provides trifluoperazine effects in combination therapy with cisplatin or gefitinib, wherein FIG. 3A shows the half maximal inhibitory concentration of the conventional chemotherapy drug cisplatin on various NSCLC spheroids (SP) and their corresponding parental cells; FIGS. 3B and 3C show the results of cell viability assay and caspase-3 activity assays, respectively, for various NSCLC spheroids treated with cisplatin (10 μM) for 24 hours; FIG. 3D shows the results of cell number measurements of CL83 and CL141 cancer spheroids after treatment with trifluoperazine in combination with cisplatin; FIG. 3E provides assessment of the combination of trifluoperazine and gefitinib by isobologram analysis, in which normalized isobolograms for EGFR-wide type (CL141) and EGFR mutation cells (CL97 and CL25) exposed to all possible drug combinations of trifluoperazine (0.5, 2.5 and 5 μM) and gefitinib (2.5, 5 and 10 μM) for 48 h are shown; symbols designate the combination index value for each fraction affected; the curves were generated by Calcusyn software to fit the experimental points; the data are representative of 3 independent experiments; values below the line are synergistic, whereas those close to the line are additive and those above the line antagonistic; FIG. 3F shows the results of cell number measurements of CL141, CL97, and CL25 spheroids treated with trifluoperazine (10 μM), gefitinib (5 μM), or both (TFP+Gef), respectively, for 48 hours; FIG. 3G provides the percentages of ALDH⁺ cells in CL141 cells, which was analyzed by flow cytometry; and FIG. 3H shows that trifluoperazine enhanced gefitinib inhibition of CL141 self-renewal; disaggregated CL141 spheroids were seeded at clonal density on low adhesion plates for secondary cancer spheroid formation. All values are the average of triplicate experiments with the S.D. indicated by the error bars (**, P<0.01).

FIG. 4 provides in vivo monitoring of trifluoperazine-mediated anti-tumor effects; wherein FIG. 4A shows representative bioluminescent images of CL97-bearing mice over the period of 4 weeks (top panel) and changes in bioluminescence intensity (BLI) were measured and plotted as fold change in BLI over time (bottom panel), in which CL97 bulk tumor cells were intravenously injected into NOD/SCID mice that subsequently received different treatments, namely vehicle (control), trifluoperazine (TFP) (5 mg/kg/day), gefitinib (150 mg/kg/day, oral gavage), and combination of gefitinib (100 mg/kg/day, oral gavage) and trifluoperazine (5 mg/kg/day, i.p); the tumor burden was measured and judged by the fold changes in bioluminescence, and ranked in decreasing order as follows: vehicle control>gefitinib>trifluoperazine>combined treatment; notably, tumor burden between mice receiving vehicle and gefitinib was not significantly different, and the tumor burden in mice which received the combined treatment was significantly lower than that of mice receiving trifluoperazine treatment (*p<0.05) and those receiving vehicle or gefitinib (**p<0.01); FIG. 4B shows representative bioluminescent images (top panel) of NOD/SCID mice, in which vehicle- and trifluoperazine-pretreated (5 μM<IC50, overnight treatment) CL97 tumor spheroids were orthotopically injected into the lung of the NOD/SCID mice for tumorigenic ability tests; in-situ tumor growth was significantly delayed and suppressed in trifluoperazine-pretreated animals (top panel), where the measurement of the tumor burden plotted as fold change in BLI (bottom panel) shows significant difference between the two groups (*p<0.05); and FIG. 4C demonstrates that samples from the combined treatment of trifluoperazine and gefitinib (Comb) provided the most significant suppression of β-catenin, c-Myc and cyclin D1 expression as compared to those from the treatment of trifluoperazine alone, gefitinib alone and vehicle control, whereas the expression level of caspase-3, a pro-apoptotic molecule, was increased in all treatment groups except for the vehicle control; similarly, β-catenin, c-Myc and cyclin D1 expression levels were suppressed in trifluoperazine-pretreated tumor spheroids while activated caspase-3 expression was increased. Total cell lysates were harvested from tumor biopsies of mice which received different treatments and their protein profiles were examined.

### DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

The phrase “eliminating cancer stem cells” as used herein refers to a process of reducing the numbers of and/or inhibiting the clonogenicity and stemness-associated markers of CSCs to an extent that the tumor initiating ability thereof can be suppressed.

As used herein, the term “anti-cancer drug” refers to any drug providing anti-cancer effect, including but not limited to gefitinib, cisplatin, Tarceva, and anti-EGFR antibody. In embodiments of the invention, the anti-cancer drug is preferably gefitinib or cisplatin.

As used herein, the term “antipsychotic phenothiazine derivatives”, “antipsychotic ” or “anti-psychotic drug” refers to a group of compounds having the structure of formula I:

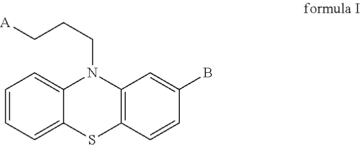

wherein the 10H-phenothiazine derivatives bear an alkyl substituent, in which A is N(CH₃)₂, a N-methyl or N-ethyl piperazinyl group, a N-(hydroxyethyl)piperazinyl group, or a N-methyl piperidinyl group, and B is SCH₃, Cl, CF₃, or H.

According to the invention, examples of the compound having the structure of formula I include but are not limited to the anti-psychotic drugs as shown in Table 1.

TABLE 1
Anti-psychotic drugs

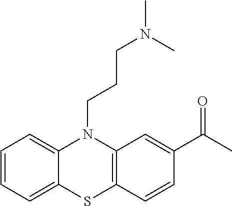 [SMILES: CC(=O)c1ccc2c(c1)N(CCCN(C)C)c1ccccc1S2]

Acepromazine

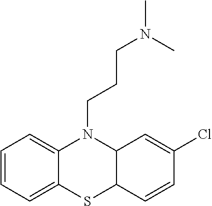 [SMILES: CN(C)CCCN1c2ccccc2SC2C=CC(Cl)=CC21]

Chlorpromazine

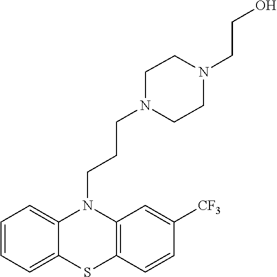 [SMILES: OCCN1CCN(CCCN2c3ccccc3Sc3ccc(C(F)(F)F)cc32)CC1]

Fluphenazine

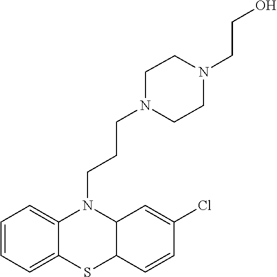 [SMILES: OCCN1CCN(CCCN2c3ccccc3SC3C=CC(Cl)=CC32)CC1]

Pherphenazine

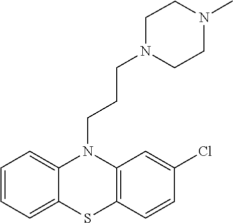 [SMILES: CN1CCN(CCCN2c3ccccc3Sc3ccc(Cl)cc32)CC1]

Prochlorperazine

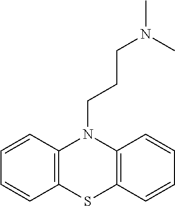 [SMILES: CN(C)CCCN1c2ccccc2Sc2ccccc21]

Promazine

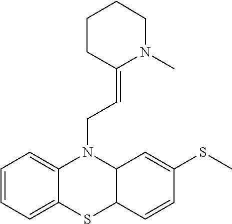 [SMILES: CSC1=CC2C(C=C1)Sc1ccccc1N2C/C=C1\CCCCN1C]

Thioridazine

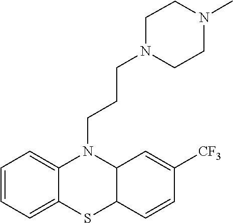 [SMILES: CN1CCN(CCCN2c3ccccc3SC3C=CC(C(F)(F)F)=CC32)CC1]

Trifluoperazine

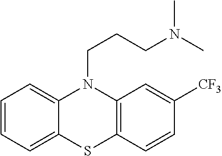 [SMILES: CN(C)CCCN1c2ccccc2Sc2ccc(C(F)(F)F)cc21]

Triflupromazine

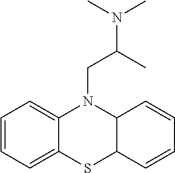 [SMILES: CC(CN1c2ccccc2SC2C=CC=CC21)N(C)C]

Promethazine

According to the invention, it was unexpectedly found that some of known antipsychotic phenothiazine derivatives have anti-CSC effects.

In this invention, CSC-like cells isolated from the CL141 cell line using side population technique were enrolled to examine the potential anti-CSC effects of some of the known antipsychotics. Table 2 summarizes the results from the MTT, side population, and clonogenic assays. Six of the antipsychotics tested, including trifluoperazine, thioridazine, chlorpromazine, perphenazine, triflupromazine and promazine, were found to reduce the percentages (>50%) of side population cells among CL141 cells (Table 2).

TABLE 2

| | MTT assay | Clonogenic assay | Reduced side |
|---|---|---|---|
| Drug Name | (IC50) (μM) | (IC50) (μM) | population |
| Trifluoperazine | >10 | 1.25~2.5 | Yes |
| Prochlorperazine | >10 | 5~10 | No |
| Thioridazine | >10 | 2.5~5 | Yes |
| Chlorpromazine | 5~10 | 2.5~5 | Yes |
| Fluphenazine | >10 | ND | No |
| Acepromazine | >10 | 1~2.5 | No |
| Promethazine | >10 | 5~10 | No |
| Perphenazine | >10 | 5~10 | Yes |
| Triflupromazine | >10 | ND | Yes |
| Promazine | >10 | ND | Yes |

ND: not determined.
MTT and clonogenic assays were performed for A549 cells, whereas the side population data were from experiments conducted on CL141 cells.

Therefore, according to the invention, the anti-psychotic drug as a cancer stem cell inhibitor may be trifluoperazine, thioridazine, chlorpromazine, perphenazine, triflupromazine and promazine.

Further in vitro and in vivo experiments demonstrated that such compounds, particularly trifluoperazine, are capable of eliminating cancer stem cells, such as lung CSCs (see Examples).

Accordingly, the invention provides use of a compound having the structure of formula I in the manufacture of a medicament for eliminating cancer stem cells (CSCs):

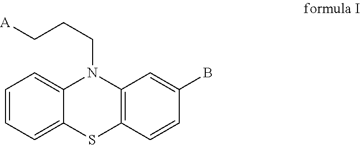

wherein A is N(CH₃)₂, a N-methyl or N-ethyl piperazinyl group, a N-(hydroxyethyl)piperazinyl group, or a N-methyl piperidinyl group, and B is SCH₃, Cl, CF₃, or H. For example, the compound having the structure of formula I may be trifluoperazine, chlorpromazine, thioridazine, perphenazine, triflupromazine, promazine or a combination thereof.

In one embodiment of the invention, the compound having structure of formula I is trifluoperazine, which has the structure of

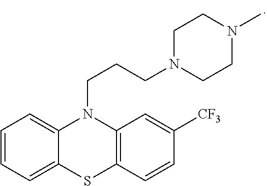 [SMILES: CN1CCN(CCCN2c3ccccc3Sc3ccc(C(F)(F)F)cc32)CC1]

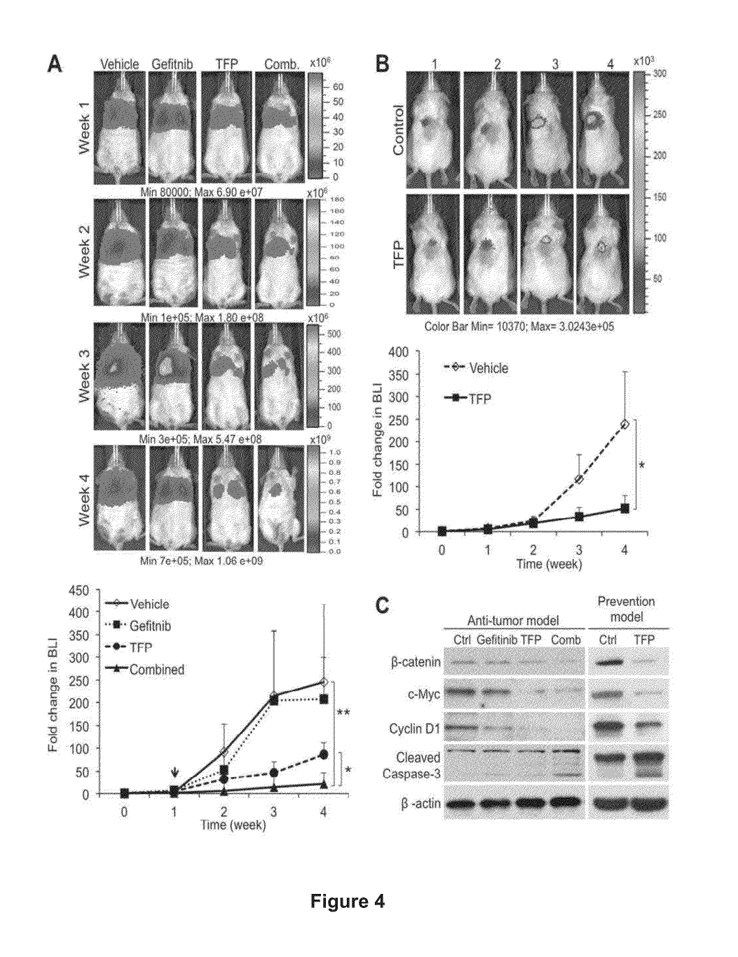

Unexpectedly, it was also found that trifluoperazine alone significantly reduced in-situ tumor growth as compared to vehicle-treated control in a prevention experiment, in which CL97-L2G cells were pre-treated with trifluoperazine before orthotopically implanted into NOD/SCID mice (FIG. 4B).

Thus, the present invention also provides a use of formula I as above mentioned in the manufacture of a medicament for preventing a cancer.

In addition, it was also confirmed in the invention that trifluoperazine in combination with an anti-cancer drug provides a synergistic effect in inhibiting the growth and/or differentiation of CSCs, and in reducing drug resistance. In one embodiment of the invention, the compound of formula I at an effective amount can be administered in combination with an anti-cancer drug to provide a synergistic effect in eliminating cancer stem cells and in reducing drug resistance of a cancer.

It is further demonstrated in the invention that trifluoperazine treatment suppressed tumorigenesis of gefitnib-resistant tumor cells in the lung cancer animal model (see Examples).

Accordingly, further provided in the invention is a method for treating a cancer in a subject resistant to standard chemotherapeutic treatments comprising administering to the therapeutically effective amount of trifluoperazine in combination of an anti-cancer drug, wherein the anti-cancer drug is administered to the subject before, simultaneously with or after the administration of trifluoperazine. In embodiments of the invention, the method can reduce the resistance to the standard chemotherapeutic treatments and inhibit the growth and/or differentiation of CSCs. The present invention also provides a pharmaceutical composition for treating a cancer in a subject resistant to standard chemotherapeutic treatments.

According to the invention, the anti-cancer drug and the trifluoperazine to be administered simultaneously may be formulated into two separate pharmaceutical compositions or one pharmaceutical composition.

As used herein, the term “effective amount” or “therapeutically effective amount” refers to an amount sufficient for eliminating cancer stem cells or reducing drug resistance of a cancer, which is depending on the mode of administration and the condition to be treated, including age, body weight, symptom, therapeutic effect, administration route and treatment time. For example, the effective amount of trifluoperazine may be 10 to 60 mg/day, preferably 20 to50 mg/day, or more preferably 35-45 mg/day.

When a cancer has progressed or returned following an initial treatment with surgery, radiation therapy, and/or chemotherapy, it is said to be recurrent or relapsed. For example, among non-small cell lung cancer (NSCLC) patients with an initial treatment with surgery, radiation therapy, and/or chemotherapy, around one third of patients are diagnosed with recurrent NSCLC. For a patient with non-small cell lung cancer, a surgical resection remains the mainstay treatment; however, the reported failure rate in stage I NSCLC ranges from 27% to 38%, and about 90% cancer deaths are associated with tumor recurrence or metastasis. In this invention, it was demonstrated that at 3 or 4 weeks after treatment in a NOD/SCID mice model bearing CL97 bulk tumor cells, both trifluoperazine alone or a combination of trifluoperazine and gefitinib significantly reduced tumor burden in the mice, whereas the treatment of genfitnib alone resulted in no effects in suppressing tumor recurrence (FIG. 4A).

Therefore, also provided in the present invention is a pharmaceutical composition for preventing or delaying cancer recurrence comprising a therapeutically effective amount of the compound of formula I, particularly trifluoperazine, and an anti-cancer drug, such as gefitnib or cisplatin. In particular, the pharmaceutical composition should be administrated to a cancer patient after an initial treatment with such as surgery, radiation therapy, and/or chemotherapy.

In the present invention, the active ingredient may be administered in any route that is appropriate, including but not limited to parenteral or oral administration. The compositions for parenteral administration include solutions, suspensions, emulsions, and solid injectable compositions that are dissolved or suspended in a solvent immediately before use. The injections may be prepared by dissolving, suspending or emulsifying one or more of the active ingredients in a diluent. Examples of said diluents are distilled water for injection, physiological saline, vegetable oil, alcohol, and a combination thereof. Further, the injections may contain stabilizers, solubilizers, suspending agents, emulsifiers, soothing agents, buffers, preservatives, etc. The injections are sterilized in the final formulation step or prepared by sterile procedure. The pharmaceutical composition of the invention may also be formulated into a sterile solid preparation, for example, by freeze-drying, and may be used after sterilized or dissolved in sterile injectable water or other sterile diluent(s) immediately before use.

The present invention is further illustrated by the following examples, which are provided for the purpose of demonstration rather than limitation.

### EXAMPLES

I. Materials and Methods

Cell Culture, Chemicals, and Clonogenic Assay

The NSCLC cancer cell lines, A549, H1975, CL25, CL83, CL97, CL141, and CL152 were maintained in RPMI medium. Tested cells were seeded respectively in 6 well plates with 10⁴ cells per well for 14 days. Each well contained 10 ml RPMI medium as cultured condition for NSCLC cells. Trifluoperazine, chlorpromazine, thioridazine, triflupromazine, and promazine were purchased from Sigma and perphenazine was from prestwick. Trifluoperazine and other tested drugs were added 24 hours after seeding of the cells. The medium and trifluoperazine were changed once on day 4. After the treatments, cells were washed with PBS, and the colonies were fixed with fix solution (acetic acid:methanol=1:3) and stained with 0.5% crystal violet in methanol. After removing the crystal violet carefully and rinse with tap water, the colonies were counted manually.

Side Population Analysis and Purification using Flow Cytometry

Single-cell suspensions of cells were detached from dishes with Trypsin-EDTA (Invitrogen) and suspended at 1×10⁶ cells/mL in Hank's balanced salt solution (HBSS) supplemented with 3% fetal calf serum and 10 mM Hepes. These cells were then incubated at 37° C. for 90 minutes with 20 μg/mL Hoechst 33342 (Sigma Chemical, St. Louis, Mo.), either alone or in the presence of 50 μmol/L verapamil (Sigma), an inhibitor of the verapamil-sensitive ABC transporter. After 90 minutes incubation, the cells were centrifuged immediately for 5 minutes at 300 g and 4° C. and resuspended in ice-cold HBSS. The cells were kept on ice to inhibit efflux of the Hoechst dye, and 1 μg/mL propidium iodide (BD) was added to discriminate dead cells. Finally, these cells were filtered through a 40 μm cell strainer (BD) to obtain single-suspension cells. Cell dual-wavelength analysis and purification were performed on a dual-laser FACS Vantage SE (BD). Hoechst 33342 was excited at 355 nm UV light and emitted blue fluorescence with a 450/20 band-pass (BP) filter and red fluorescence with a 675 nm edge filter long-pass (EFLP). A 610 nm dichroic mirror short-pass (DMSP) was used to separate the emission wavelengths. PI-positive (dead) cells were excluded from the analysis.

Soft Agar Assay

Freshly sorted CL141 side population (SP) and non-side population (NSP) cells were counted and plated in triplicate at 200 cells per well in six-well plates coated with 1% agarose. Anchorage-independent growth was assessed after incubation for 10-14 days in culture media with or without trifluoperazine (0, 5 and 10 μM), which was replaced every 4 days. Plates were stained with 0.005% crystal violet, and the colonies were counted manually under a microscope and photographed.

Tumor Spheroid Assay

For the formation of tumor spheroids, cells were cultured in HEScGRO serum-free medium (human) (Chemicon) supplemented with 20 ng/mL Hegf, 10 ng/mL hFGF-b and NeuroCult NS-A proliferation supplements. Cells were seeded at low densities (1000 cells/mL) in 12-well low adhesion plates at 1 mL per well. Spheroids (tight, spherical, nonadherent masses >90 μm in diameter) were counted, and at least 50 spheroids per group were measured with an ocular micrometer. For secondary spheroid-forming assays, primary spheroids were dissociated mechanically and processed as in the primary assay. For the quantification of the percentage of spheroid-forming cells, cells were seeded at one cell per well in 96-well plates.

Reporter Assay

Spheroid cells were plated in 6-well plates, grown to 80%-90% confluence, and transiently transfected with 1.8 μg TOPflash or FOPflash plasmids using Lipofectamine. TOPflash contains 3 copies of the Tcf/Lef binding sites upstream of a thymidine kinase (TK) promoter and the firefly luciferase gene. FOPflash contains mutated copies of the Tcf/Lef sites and is used as a control for measuring nonspecific activation of the reporter. To normalize for transfection efficiency, cells were cotransfected with 0.2 μg of the internal control reporter encoding Renilla reniformis luciferase driven by the TK promoter. After transfection, cells were incubated in medium with or without trifluoperazine (0-10 μM) for 48 hours and then lysed with reporter lysis buffer after harvest. Luciferase activity was determined by the Luciferase Assay System (Promega) using a Microplate Luminometer (Berthold). The experiments were performed in triplicate, and the results were reported as fold induction compared with the control group after transfection efficiency normalization.

Aldefluor Assay

High aldehyde dehydrogenase (ALDH) enzyme activity was used to detect lung CSC populations in this study. The Aldefluor assay was performed according to the manufacturer's guidelines (StemCell Technologies). Briefly, single cells obtained from cell cultures were incubated in an Aldefluor assay buffer containing an ALDH substrate (bodipy-aminoacetaldehyde, BAAA) for 50 minutes at 37° C. As a negative control, a fraction of cells from each sample was incubated under identical conditions in the presence of an ALDH inhibitor (diethylaminobenzaldehyde, DEAB). Flow cytometry was used to measure the ALDH-positive cell population.

Western Blotting Analysis

Cells were lysed in lysis buffer (50 mM Tris-HCl, pH 7.4, 5 mM MgCl₂, 1% Nonidet P-40, 150 mM NaCl, 1 mM phenylmethylsulfonyl fluoride). Total protein was isolated and subjected to SDS polyacrylamide gel electrophoresis and electrotransfered onto PVDF membranes (Millipore). Primary antibodies Bax, Bak, Bcl-2, XIAP, Mcl-1, Cleaved Caspase 9, Caspase 3, PARD, c-Myc, CD44, cyclin D1 were obtained from Cell Signaling, Met was purchased from Santa Cruz and CD133 was from Miltenyi Biotec, and secondary antibodies for anti-mouse and anti-rabbit horseradish preoxidase (HRP)-conjugation were from Chemicon International. The protein detection was performed with enhanced chemiluminescence (ECL™) method captured by a Luminescence Imaging System (LAS-4000™, Fuji Photo Film Co., Ltd).

Generation of a Stable Dual Reporter-Expressing Lung Cancer Cell Line

The dual optical reporter system L2G fusion construct (firefly luciferase 2 and eGFP) was a generous gift from Dr. Gambhir, Stanford University. Stable L2G-expressing CL97 cells were generated accordingly. Briefly, CL97 cells with stable integration of the L2G reporter were generated by lentiviral-mediated gene transfer. 293FT cells were transfected with the lentiviral vector L2G, the packaging plasmid pCMVΔ8.74 and the envelope plasmid pMD2.G (Nat Biotechnol 1997; 15:871-875). The target CL97 cells were infected with the viral particles and selected using Zeocin. CL97 cells carrying the L2G reporter system (CL97-L2G) were obtained and expanded for further experiments.

Evaluation of Trifluoperazine's Anti-CSC Effects using Non-Invasive Bioluminescent Imaging

NOD/SCID mice were purchased from National Taiwan University and maintained in compliance with the institutional policy. All animal procedures were approved by the IACUC (Institutional Animal Care and Use Committee) at Taipei Medical University.

For bulk lung tumor model, CL97-L2G cells were intravenously administered into the animals via tail vein at a concentration of 1×10⁶ cells/100 μl PBS. One week post tumor injection, different treatment regimens were started. Four regimens were performed, trifluoperazine (5 mg/kg/day), gefitinib (150 mg/kd/day, oral gavage) and a combination of trifluoperazine (5 mg/kg/day i.p injection)+gefitinib (100 mg/kg/day, oral gavage) for a period of 4 weeks.

To examine the preventive and anti-CSC effects of trifluoperazine, CL97-L2G spheroids were pre-treated with trifluoperazine (5 μM, <IC50, overnight), re-suspended from their spheroid form and orthotopically injected into the lungs of NOD/SCID mice (1×104 cells/50 μL matrigel/inoculation). The animals did not receive further treatment for the span of the experiment. CL97-L2G-bearing mice (both bulk lung tumor and CSC models) were imaged weekly using the IVIS 200 system (Caliper Life Sciences). Data are expressed as fold change in total photon flux/initial total photon flux and were analyzed using Living Image 1.0 software (Caliper Life Sciences). Mice were humanely sacrificed at the end of experiments and lung tumor biopsies were obtained for further analysis.

II. Results

Trifluoperazine Inhibited Proliferation and Induced Apoptosis of Gefitinib-Resistant NSCLC Cells

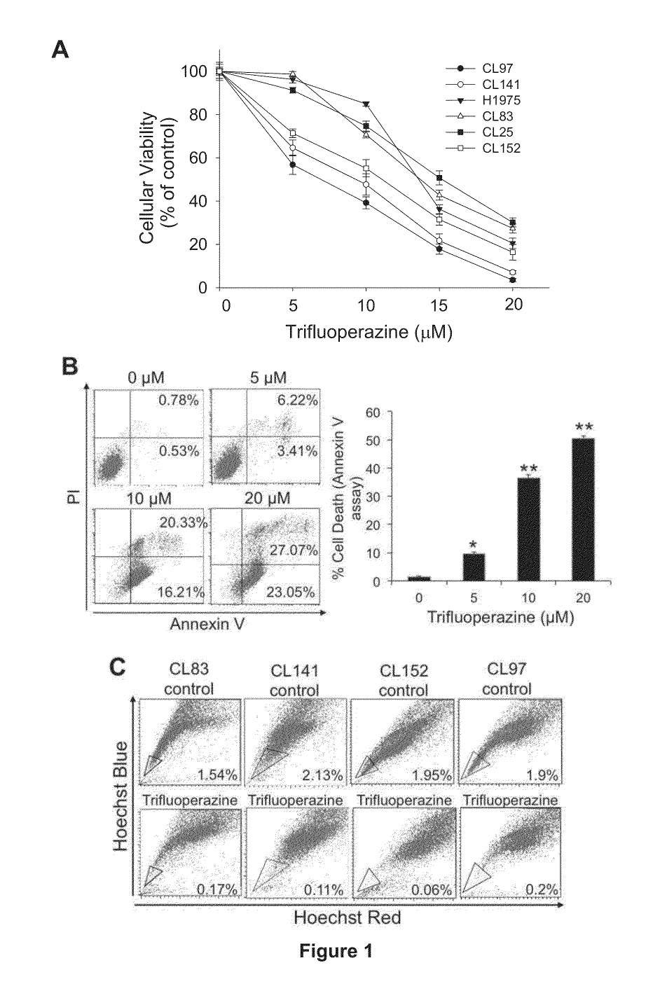

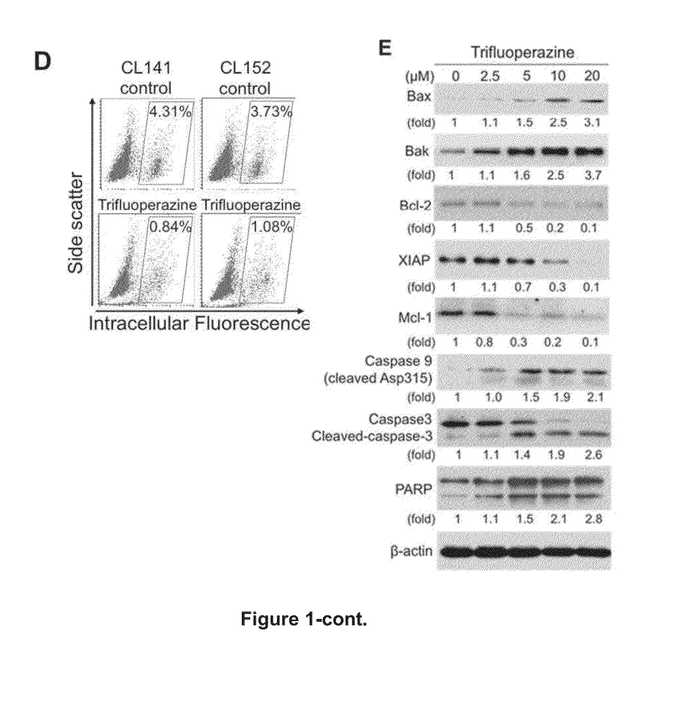

We hypothesized that trifluoperazine would inhibit tumor growth and overcome drug resistance by exerting anti-CSC effects. In addition to the commonly used cell lines (A549 and H1975), we also established several cell lines including CL83, CL141, CL152, CL25, and CL97 cell lines which were isolated from the pleural effusion of NSCLC patients at the National Taiwan University Hospital. The investigation was approved by the Institutional Review Board of the National Taiwan University Hospital. Informed consent was obtained before pleural effusion was collected. A summary of the main features of these cell lines, including their histologic and mutational characteristics, as well as whether they have intrinsic or acquired resistance to EGFR TKIs, is provided in Table 3. We demonstrated that trifluoperazine dose-dependently inhibited NSCLC cell growth, and the respective IC₅₀ values (48 hour incubation) for CL83, CL141, CL152, CL25, CL97, and H1975 were 14, 8.5, 12, 13, 7.2, and 15 μM, respectively (Table 3 and FIG. 1).

TABLE 3
The clinical characteristics, gene mutations, and responses to EGFR-TKI and
trifluoperazine for the non-small cell lung cancer cell lines in this study.

| | | | | EGFR | PTEN | p53 | KRA5 | Resistance | IC50 | | IC50 | |
|---|---|---|---|---|---|---|---|---|---|---|---|---|
| Cell | | | | mutation | mutation | mutation | mutation | to | for | | for | |
| line | Gender | Histology | Clinical information | status | status | status | status | EGFR-TKI | gefitinib | | trifluoperazine | |
| CL83 | Male | adenocarcinoma | Collected on the 16th day | WT | Normal | ND | WT | intrinsic | >10 | μM | 14 | μM |
| | | | after gefitinib treatment, | | | | | resistance | | | | |
| | | | disease progression | | | | | | | | | |
| CL141 | Male | adenocarcinoma | Collected while chemonaïve | WT | Loss | R248W | WT | intrinsic | >10 | μM | 8.5 | μM |
| | | | | | | | | resistance | | | | |
| CL152 | Male | squamous cell | Collected while Chemonaïve | WT | Loss | R248W | WT | intrinsic | >10 | μM | 12 | μM |
| | | carcinoma | | | | | | resistance | | | | |
| CL25 | Male | adenocarcinoma | Collected before erlontinib | Exon 19 | Normal | C135Y | WT | sensitive | 50 | nM | 13 | μM |
| | | | treatment, partial response | deletion | | | | | | | | |
| CL97 | Male | adenocarcinoma | Collected after erlontinib and | G719A/ | Normal | R273H | WT | acquired | >10 | μM | 7.2 | μM |
| | | | several cycles of | T790M | | | | resistance | | | | |
| | | | chemotherapy, response to | | | | | | | | | |
| | | | erlotinib use: disease | | | | | | | | | |
| | | | progression | | | | | | | | | |
| H1975 | Female | adenocarcinoma | established in July 1988 | L858R/ | Normal | WT | WT | intrinsic | >10 | μM | 15 | μM |
| | | | from a non-smoker | T790M | | | | resistance | | | | |
| A549 | Male | adenocarcinoma | Initiated in 1972 by D.J. | WT | Normal | WT | G125 | intrinsic | >10 | μM | >10 | μM |

| | |
|---|---|
| Giard, et al. through explant | resistance |
| culture of lung | |
| carcinomatous tissue from a | |
| 58-year old Caucasian. | |

WT: wild type;
EGFR-TKI: epidermal growth factor receptor-tyrosine kinase inhibitor.
The clinical information of H1975 and A549 were obtained from ATCC.
*ND: not determined.

Among these cell lines, we chose CL141, an adenocarcinoma with wild-type EGFR status which shows resistance to gefitinib, as a representative target cell line for apoptosis analysis. Annexin V/PI staining was performed after treatment with different dosages of trifluoperazine. Both early and late apoptotic cells were counted. After 48 hours, trifluoperazine-treated CL141 cells exhibited a dose-dependent increase in Annexin V-positive cells when compared to the control cells (FIG. 1B). The results indicated that trifluoperazine inhibited the proliferation of and induced apoptosis of gefitinib-resistant NSCLC cells.

Trifluoperazine Reduced the Percentage of and Induced Apoptosis of Lung CSCs

We selected gefitnib-resistant cell lines CL83, CL141, CL152 (with wild-type EGFR) and CL97 (harboring EGFR-G719A+T790M mutations) and isolated their CSCs using side-population method (1.54%, 2.13%, 1.95%, and 1.9% of the side population cells, respectively). After treatment with 5 μM trifluoperazine, the percentage of side population cells significantly decreased (FIG. 1C).

For further clarification, we examined if trifluoperazine treatment could deplete the percentage of the cells with ALDH expression, an established marker for both hematopoietic and NSCLC CSCs. CL141 (adenocarcinoma) and CL152 (squamous cell carcinoma) were selected as representative target NSCLC cell lines. Trifluoperazine treatment decreased the ALDH⁺ CL141 cell population from 4.31% to 0.84%, and from 3.73% to 1.08% in CL152 cells (FIG. 1D).

To investigate the apoptotic-associated signal transduction in lung CSC after trifluoperazine treatment, CL97 (adenocarcinoma with EGFR-T790M-acquired resistance mutation) was selected as a target cell line. After trifluoperazine treatment of CL97 cancer spheroids, Bax, Bak, cleaved PARP, caspase-3, and caspase-9 was increased dose-dependently, whereas anti-apoptotic Bcl-2, XIAP, and Mcl-1 were decreased (FIG. 1E).

Trifluoperazine Inhibited the Clonogenicity and Stemness-Associated Markers of Lung CSCs

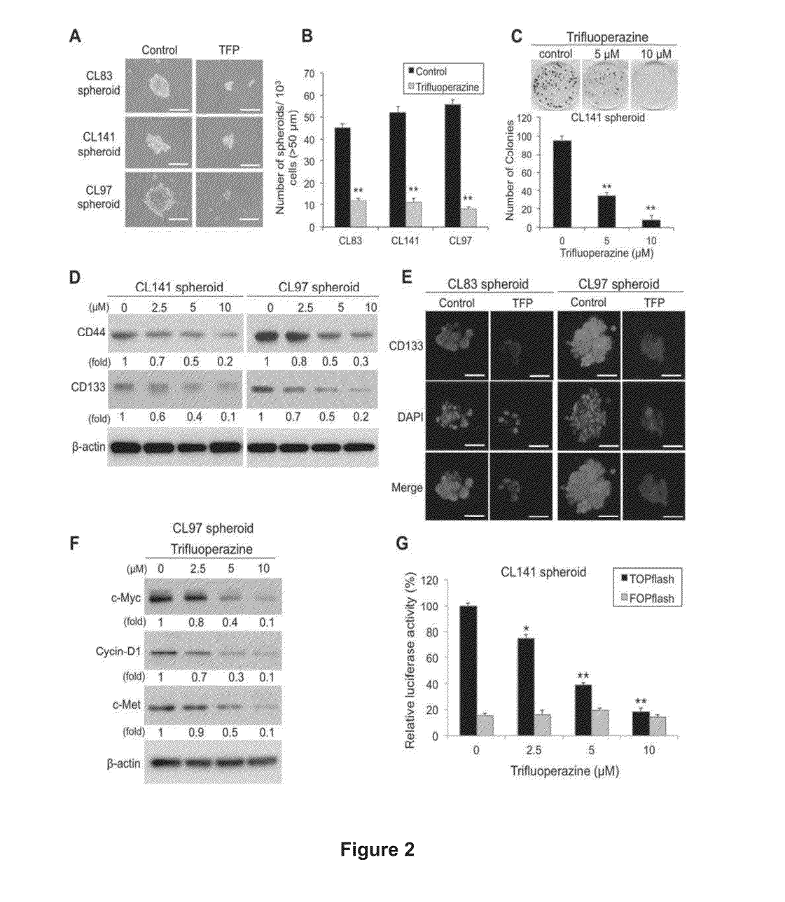

Three different gefitinib-resistant lung CSCs, including CL141 (wild-type EGFR), CL83 (wild-type EGFR) and CL97 (EGFR-G719A+T790M acquired resistance mutation) were treated with trifluoperazine to examine its effects on tumor spheroid formation. Trifluoperazine dose-dependently decreased the size and number in all spheroids (FIGS. 2A, 2B, and 2C). The mean colony formation of CL141 spheroids on soft agar decreased after 12 days of treatment with either 5 or 10 μM trifluoperazine (FIG. 2C, mean colony number, control: 92, 5 μM: 32, 10 μM: 8). CL141 and CL97 spheroids were treated with increasing dosages of trifluoperazine (0, 2.5, 5, and 10 μM) for 48 hours. Two established lung CSC markers, CD44 and CD133, were dose-dependently down-regulated by trifluoperazine as measured by Western blotting and immunochemical staining (FIGS. 2D and E).

To explore the molecular mechanisms mediated by trifluoperazine, CL97 spheroids were treated with trifluoperazine and analyzed by western blots. Wnt/β-catenin signaling downstream targets, Cyclin D1 and c-Myc, and c-Met were decreased by trifluoperazine (FIG. 2F). Additionally, trifluoperazine (at low concentration, 2.5 μM) inhibited TCF-mediated transcription in CL141 spheroids disrupted spheroid formation (FIG. 2G).

Trifluoperazine Synergistically Inhibits Lung CSCs in Vitro while Combined with Cisplatin or Gefitinib

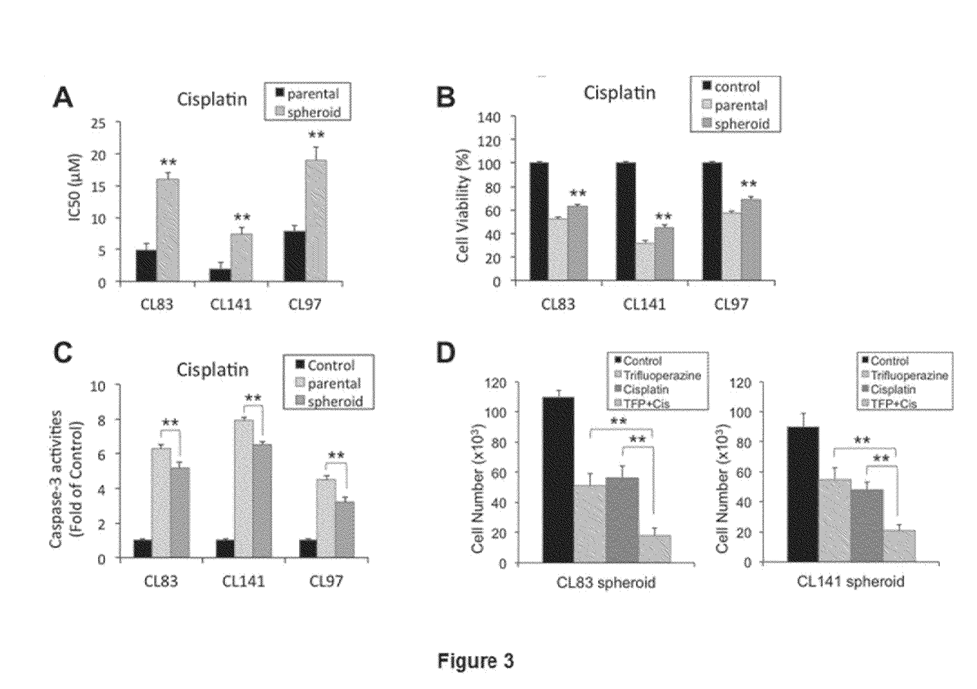

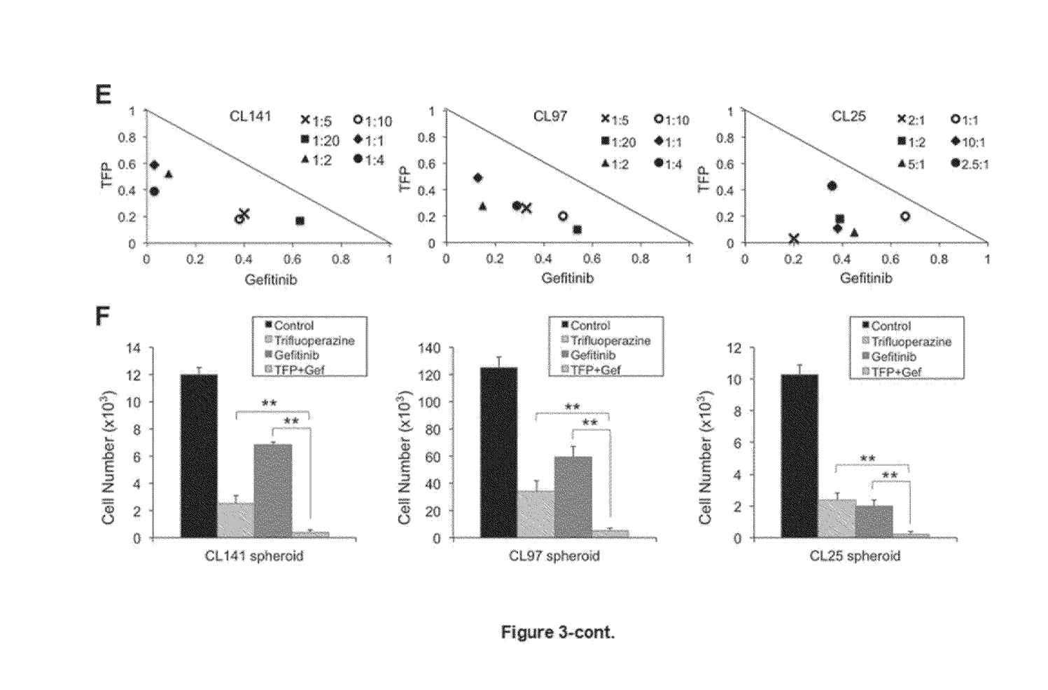

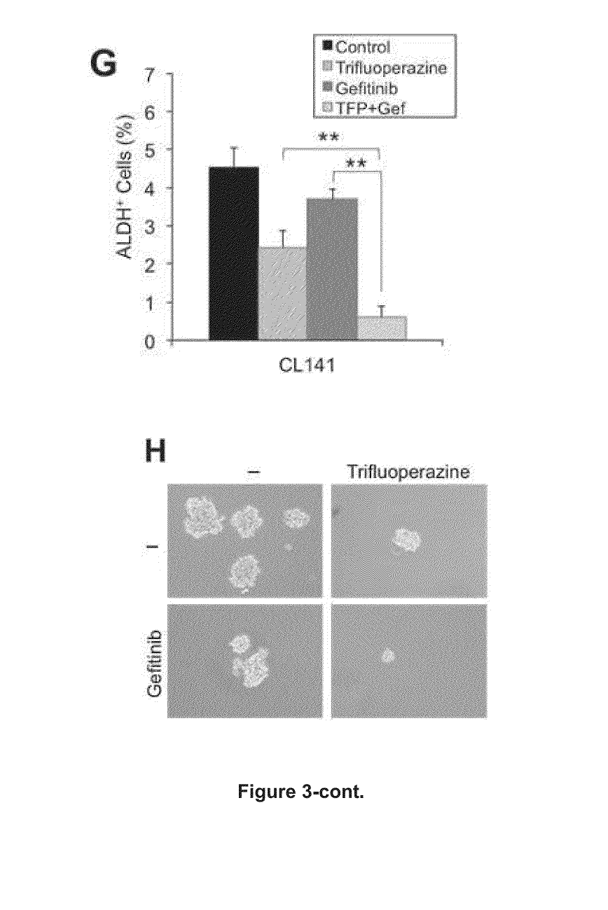

We selected three gefitinib-resistant NSCLC cell lines, CL141 (wild type EGFR), CL83 (wild type EGFR) and CL97 (EGFR-G719A+T790M acquired resistant mutation) to determine if trifluoperazine could sensitize these cells towards chemotherapeutic agents. While treating with 10 μM of cisplatin for 24 hours, all CL141, CL83 and CL97 spheroids showed a significantly higher IC₅₀ (FIG. 3A) than their parental cells. Under the same condition, all spheroids showed higher viability and a lower caspase-3 activity as compared to their parental cells (FIGS. 3B and 3C).

Next, we examined whether trifluoperazine could enhance the cytotoxic effects of cisplatin or gefitinib. The combined trifluoperazine and cisplatin treatment provided a significantly higher cytotoxic effect in both CL83 and CL141 spheroids than either trifluoperazine or cisplatin treatment alone (FIG. 3D).

Assessment of the combinatorial activity of trifluoperazine and gefitinib was performed using the isobolographic method (Chou T C and Talalay P. Adv Enzyme Regul 1984; 22:27-55). Values below the line are synergistic, whereas those close to the line are additive and those above the line antagonistic. The synergistic activity of both agents was demonstrated from the normalized isobolograms obtained from the EGFR-wide-type cells (CL141), EGFR-G719A+T790M mutation cells (CL97) and EGFR-exon 19 deletion cells (CL25) (FIG. 3E). The enhanced cytotoxicity was also observed in all CL141, CL97 and CL25 spheroids. To investigate the effect of trifluoperazine on gefitinib therapy, CL25 (EGFR-TKI sensitive cell line) spheroids growth inhibition assay was performed as a positive control. CL25 spheroids were exposed to individual agents or a combination of trifluoperazine with gefitinib, as well as CL141 and CL97 cell lines (FIG. 3F). Gefitnib alone effectively suppressed the spheroid formation in CL25 but significantly less in CL141 and CL97 cells. The combination of trifluoperazine and gefitnib significantly suppressed the spheroid formation of CL141 and CL97. These observations indicated that the addition of trifluoperazine sensitized gefitinib-resistant lung cancer cells. In addition, the percentage of ALDH+CL141 cells was moderately decreased at 10 μM of trifluoperazine. However, an enhanced inhibitory effect was observed when trifluoperazine was combined with 5 μM of gefitinib (FIG. 3G). A similar enhanced inhibition on CL141 spheroid formation was observed (FIG. 3H).

Trifluoperazine Treatment Suppressed Tumorigenesis of Gefitinib-Resistant CL97-L2G in Mouse Lung Cancer Models

NOD/SCID mice bearing gefitinib-resistant CL97-L2G (G719A+T790M acquired resistance mutation) cells were used to evaluate the anti-tumor effects of trifluoperazine. First, CL97 bulk tumor cells were injected intravenously into the tail vein of NOD/SCID mice that subsequently received vehicle with trifluoperazine alone (5 mg/kg/day, i.p), gefitinib alone (150 mg/kg/day, oral gavage), or a combination of trifluoperazine (5 mg/kg/day, i.p) and gefitinib (100 mg/kg/day, oral gavage) treatment. Comparatively, mice that received trifluoperazine alone showed significantly lower tumor burden than those that received vehicle and gefitinib alone (FIG. 4A). As expected, gefitinib-treated mice demonstrated a similar level of tumor burden as the vehicle control group. Mice that received the gefitinib/trifluoperazine combined treatment exhibited the lowest tumor burden. Tumor burden was measured and quantified based on the fold change in bioluminescence intensity.

In the prevention experiment, CL97-L2G cells were pre-treated with vehicle or trifluoperazine (5 μM, <IC₅₀) and orthotopically implanted into NOD/SCID mice. Mice that received the trifluoperazine-pretreated CL97-L2G cells exhibited delayed and significantly reduced in-situ tumor growth as compared to vehicle-treated control (FIG. 4B). To explore the molecular mechanisms mediated by trifluoperazine, total protein lysates were harvested from tumor samples. The expression level of stemness molecules including c-Myc and β-catenin was found to be decreased. Cyclin D1 expression was also suppressed by both trifluoperazine and the combined treatment while the activated form of caspase 3 was increased by both trifluoperazine and the combined treatment (FIG. 4C). Gefitinib treatment did not significantly influence the expression level of either c-Myc or β-catenin.

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

What is claimed is:

1. Use of a compound having the structure of formula I in the manufacture of a medicament for eliminating cancer stem cells (CSCs): 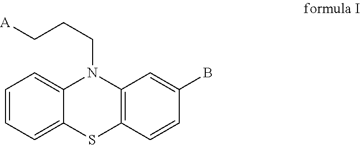
wherein A is N(CH₃)₂, a N-methyl or N-ethyl piperazinyl group, a N-(hydroxyethyl)piperazinyl group, or a N-methyl piperidinyl group, and B is SCH₃, Cl, CF₃, or H.

2. The use of claim 1, wherein the compound is trifluoperazine, chlorpromazine, thioridazine, perphenazine, triflupromazine, promazine or a combination thereof.

3. The use of claim 2, wherein the compound is trifluoperazine.

4. The use of claim 1, wherein the CSCs are lung CSCs.

5. The use of claim 4, wherein the lung CSCs are non-small cell lung cancer stem cells.

6. A pharmaceutical composition for treating a cancer comprising a therapeutically effective amount of a compound having the structure of formula I and an anti-cancer drug: 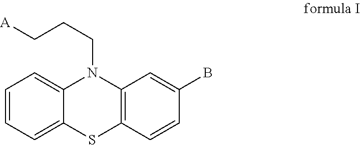
wherein A is N(CH₃)₂, a N-methyl or N-ethyl piperazinyl group, a N-(hydroxyethyl)piperazinyl group, or a N-methyl piperidinyl group, and B is SCH₃, Cl, CF₃, or H.

7. The pharmaceutical composition of claim 6, wherein the compound is trifluoperazine.

8. The pharmaceutical composition of claim 6, wherein the compound and the anti-cancer drug are in the form of two separate formulations or in the form of one formulation.

9. The pharmaceutical composition of claim 6, wherein the anti-cancer drug is gefitnib or cisplatin.

10. The pharmaceutical composition of claim 6, which is effective for eliminating CSCs.

11. The pharmaceutical composition of claim 6, wherein the cancer is lung cancer.

12. The pharmaceutical composition of claim 11, wherein the lung cancer is non-small cell lung cancer.

13. A pharmaceutical composition for preventing or delaying cancer recurrence comprising a therapeutically effective amount of a compound having the structure of formula I and an anti-cancer drug: 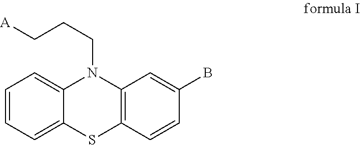
wherein A is N(CH₃)₂, a N-methyl or N-ethyl piperazinyl group, a N-(hydroxyethyl)piperazinyl group, or a N-methyl piperidinyl group, and B is SCH₃, Cl, CF₃, or H.

14. The pharmaceutical composition of claim 13, wherein the compound is trifluoperazine.

15. The pharmaceutical composition of claim 13, wherein the compound and the anti-cancer drug are in the form of two separate formulations or in the form of one formulation.

16. The pharmaceutical composition of claim 13, wherein the anti-cancer drug is gefitnib or cisplatin.

17. The pharmaceutical composition of claim 13, which is effective for CSCs.

18. The pharmaceutical composition of claim 13, wherein the cancer is lung cancer.

19. The pharmaceutical composition of claim 18, wherein the lung cancer is non-small cell lung cancer.

20. The pharmaceutical composition of claim 13, wherein the pharmaceutical composition is to be administered to a subject in need thereof after an initial cancer treatment.

21. The pharmaceutical composition of claim 20, wherein the initial cancer treatment is a surgery, radiation therapy, chemotherapy or combination thereof.

22. Use of a compound having the structure of formula I in the manufacture of a medicament for preventing a cancer: 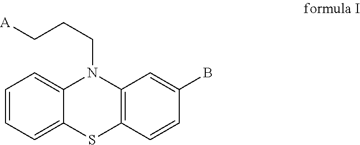
wherein A is N(CH₃)₂, a N-methyl or N-ethyl piperazinyl group, a N-(hydroxyethyl)piperazinyl group, or a N-methyl piperidinyl group, and B is SCH₃, Cl, CF₃, or H.

23. The use of claim 22, wherein the compound is trifluoperazine, chlorpromazine, thioridazine, perphenazine, triflupromazine, promazine or a combination thereof.

24. The use of claim 23, wherein the compound is trifluoperazine.

25. The use of claim 22, wherein the cancer is lung cancer.

26. The use of claim 25, wherein the lung cancer is non-small cell lung cancer.
